(19) United States
(12) Patent Application Publication
(10) Pub. No.: US 2014/0182592 A1
(43) Pub. Date: Jul. 3, 2014

(54) DEVICE AND METHOD FOR REAL TIME COUGH SOOTHING AND SUPPRESSION

(71) Applicant: Dan Aharoni, Tel Aviv (IL)

(72) Inventor: Dan Aharoni, Tel Aviv (IL)

(21) Appl. No.: 14/129,507

(22) PCT Filed: Jun. 25, 2012
(86) PCT No.: PCT/IL2012/000257

§ 371 Date: Feb. 11, 2014

(30) Foreign Application Priority Data
Jun. 26, 2011 (IL) 213777

(51) Int. Cl. A61M 16/00; A61M 16/06

(52) U.S. Cl. CPC A61M 16/0045; A61M 16/06
USPC 128/205.25

(57) ABSTRACT

A real time cough soothing and suppression device (10) formed as an elongated cylindrical object or semi-elliptic small pouch. It is preferably made of cloth or Lyocell synthetic fiber stitched to form a space (12) that is preferably filled up with polyester fibers (18). Said fibers may be soaked by liquids such as antiseptic solutions, medical lotions or breath cooling and pain relieving liquids such as Eucalyptus or Menthol.

### FIELD OF THE INVENTION

The present invention, the subject matter of this application, is found in the field of soothing and suppressing painful coughing in general, and real time soothing and verily alleviating painful and irritating harsh coughing spells in particular.

### BACKGROUND OF THE INVENTION

The inventor, like the majority of the human race, suffers at times from colds—accompanied by coughing to a lesser or a more serious degree. He is familiar with the cures—pills and syrups—and knows that eventually after several days (even weeks) the cold abates and eventually passes. Most of the treatments are aimed at curing the disease—and in due course it is healed (cured). The time it takes depends on a variety of variables, such as the person's current health state and record (history), environmental conditions, the person's background and etc. At times just inhaling dirt from the air evokes similar coughing. In the interim—the patient suffers pain and discomfort in the palate and throat when coughing, pain that gets worse when coughing spells (or series) follow one after the other, or even gets worse as the rate of following coughs increases. The subject matter of the present invention is exactly this—the invention is aimed of and provides in real time an answer to reducing (significantly assuaging) the pain, itch and discomfort in the palate and throat associated with the involuntary coughing effort caused by the coughing spells.

Before venturing to describe prior art and the present invention (also pointing how it differs from current day treatment methods) and insufficient answers by approaches and patents, let's get acquainted with the biological and physical properties of “coughing”, that causes the encountered irritation and pain.

In order to enable the reader to better understand and grasp the mechanism attending the coughing incidence and the performance of the innovative Cough-EZ device that will be introduced as the subject matter of the present invention, we submit below a short treatise about the nature of coughing and its properties. It is good to remember that there are many different types of coughing bouts. In most cases specific maladies are linked to given coughing occurrences. The action of the Cough-EZ devices provides immediate relief for those different types of coughing which is soothing, alleviating or drastically reducing the pain and irritation co-habiting with the coughing rush is achieved in all cases.

The following treatise is partly based on the Wikipedia and other sources as well as on “personal knowledge” and study.

Coughing is a reflex action started by simulation of certain sensory nerves in the lining of the respiratory passages, the tubes we use to breath. The more a person coughs—the more the irritation, hence an urge to cough follows (continues) and the pain increases. The air driven out of the lungs by coughing is released to the environment or against a palm or a paper tissue or, as some do, unto the inner area of the elbow (neither the palm nor the tissue or the inner area of the elbow are suitable for suppressing the cough). When a person coughs, there is a short intake of breath and the larynx closes momentarily. Then the abdominal and chest muscles (used for breathing) contract—which in turn increases the pressure needed to drive air out of the lungs when the larynx reopens. A blast of air occurs—coming out at a high speed, scrubbing and clearing the airway of dust, dirt or excessive secretions.

Normally, the lungs and the (lower) respiratory passages are clean. However, if dust or dirt that could become a breeding ground for bacteria gets into the lungs (and causes infections), the coughing reflex strives to clean them. Coughing is a symptom (generally not an illness)—if the reason for coughing (that needs to be treated) is not treated—coughing would continue for extended periods, increasing the irritation in the throat and culminating in “a painful coughing experience”. It is important to note that most pharmaceutical medications do not induce immediate relief. The primary purpose of the cough suppression device (the Cough-EZ), namely the subject matter of the present invention, is exactly this: to minimize in real time these painful and irritating conditions (and prevent worsening conditions), hence to lead to a quicker solution of the coughing attack while it is on, and to feel relieved and better—sooner.

### PRIOR ART

There are many instances in the literature, and many patents that present a variation of face masks as well as syrups and other potions for the coughing person. The following does not constitute a part of the present patent application, but is presented herein for the completeness of our presentation and to provide a background to statements we will introduce in this application.

Vail III et al, U.S. Pat. No. 7,048,953 (May 2006) discusses a method to reduce risks of infection of the human respiratory system by respiratory pathogens (for a person or more in an enclosed public area) by a complicated preventive procedure in essence, by inhaling concentrated vapors from an essential oil that possesses anti-pathogenic properties for ca 30 minutes before entering that area, and continue inhaling them intermittently after entering said area. This is a complicated procedure and is mentioned here only due to it being remotely similar to the use of vapors suggested in our invention—as will be described later on when presenting our invention.

A similar implementation is found in Provisional Patent Application 60/328,6912 (“Methods & Apparatus to Prevent, Cure etc.) infection to Human Respiratory System”; we also mention Provisional Patent Application 60/449,379 wherein “Hand Held Inhalers” are cited, reminiscences of our innovative invention. However, the differences between it and the innovation presented in our approach is—that we claim an invention that is meant to ease the pain and relief the attending irritation and stress of repeated cough spells in real time, namely while the coughing discomfort is felt (this point would be explained and become clear, lucid and obvious, by the treatment of the subject in our “Detailed Description of a Preferred Embodiment of the Invention” chapter).

Referring now to the subject matter of the present patent application, we note that a factor that is verily contributing to the relief effect when using a cylindrical Cough-EZ device or a pouch like Cough-EZ device (see: “Summary of the Present Invention”), is materialized by exploiting the effect generated by the compression of air in the mouth, upper throat and the lower larynx due to the intensity and the velocity of the compressed air (energy plus heat) of the fast flow of air that rushes from the coughing person that causes the absorbing material (e.g., polyester fibers) to both repel and return back (somewhat like an echo) the energy—a combined action state of affairs that at this situation results in the relief that the coughing person feels in the upper throat and lower larynx spaces.

### SUMMARY OF THE PRESENT INVENTION

As we have shown and cited above, in the period that preceded this present invention, there were many works and patents that treated face masks over the mouth front area of the face, others did cover the nose and mouth area. The declared goal of all those face mask, was to have them serve as a filter—for breathing filtered air without inhaling “bad”, contaminated or polluted air (i.e. floating dust).

Even when mentioned in conjunction with colds, flu and attending coughing, it was only for protecting people from being infected by the flu or other throat and lungs infections. Very few mentioned also the other direction of the filtering—namely—preventing the sick person from spreading contagious pathogens (bacteria, germs etc.) to the environment when coughing.

In contradistinction, the subject matter of the present invention, as was pointed above and as would be discussed factually and fully in the “Detailed Description of Preferred Embodiments” chapter, is to help the coughing person by providing immediate relief (and concurrently contribute to a curing effect) when the coughing spell is occurring, by actually reducing the pain and abating the inconvenience and irritation of the suffering person as stated in the patent application title: “Cough Soothing and Suppression”.

For this end, we developed, designed and produced a device, herein after referred to as “the Cough-EZ Device”—which is the subject matter of the present patent application, wherein its purpose is to minimize the painful and irritating conditions prevailing when coughing and leading to a quicker solution of coughing spells discomfort.

The present invention, the subject matter of this application, is materialized by implementing a device—the “Cough-EZ” device as said, described further below and with its accompanying figures and examples. As said above, the two major embodiments consist of two kinds of embodiments—a semi cylindrical (relatively long) device, preferably ca. 13 cm high, and a small cushion like semi-eliptic pouch (other structural and operational variations, that do not deviate from the scope of the Cough-EZ devices as being covered by this patent application, will also be introduced below).

In a way (but verily it is a wrong impression) a reader might think that the soothing Cough-EZ pouch devices are apt to be considered another facet of the face masks mentioned above, but actually they are significantly different, due to the materials of which they are made, the way they are filled up with polyester strands (fibers), the calculations that led to selecting the various materials that are used, their densities—and as for the polyester—the way of inserting them, the selection of different weights and “crowding” (thus selected density)—alt of which contribute to the result—which characterizes them, namely successfully fulfilling their goal of real time reduction of the pain and irritation that commonly and naturally are part of the coughing spell. These statements and claims will be easy to grasp when the structural embodiment would be presented—as per materials, layout, properties and performance, all tested and verified.

Let's consider the following additional attributes—
(a) A beneficial outcome, inherent in the use of this device in accordance with this invention (which happens to be a synergism of the method of producing the Cough-EZ device), is, as said, that usage of this device substantially reduces the volume of the coughing sounds accompanying severe (and even medium) coughing sounds—see Table No. 3.
   Measurements were conducted as per traditional acoustics processes. Accordingly, it was tested and found (table No. 3) that in fact these devices actually and substantially reduce the unwelcome noise and interference/s raised by coughing in many public events with audiences, and worse yet—in public halls like concert halls or theatres—the dreaded inevitable “music” of the winter (not only)—that only few people are considerate enough not to attend them when they have a cold—or worse.
(b) At this point, it is important to consider the immediate (real time) relief as it helps the parent whose heart aches when her/his baby or toddler coughs and cries incessantly and he can hardly help. By holding the preferably pouch device rather near the kid's mouth (or strapping it momentarily across older kids face)—the relief that follows is god sent to kids and parents simultaneously (for “the small ones”, it is preferred and more convenient to use the smaller semi-eliptic pouch device rather than the elongated cylindrical one).
   Let's remember that in the period that preceded this invention, there were a host of medicines, syrups and the like, used to cure the cold or cough bouts (as they did claim) but none to offer anything like immediate relief. Relating to the immediate relief from the coughing pain and irritation as said above, and the synergetic effect that we discussed (see measured values of noise reduction quoted in table No. 3) of the reduction of the cough “audio effects” (unwelcome loud noise) applies thus also to babies and toddlers and their parents.
(c) Both devices have a free space formed inside them. The cylinder, as well as the small pouch Cough-EZ devices (FIGS. 1, 3 respectively) provide the space for filling up with polyester fibers. Filling up the internal free space of the devices is done with fibers of various sizes—see dimensions and other parameters, table No. 2. In order to select beneficial specific weight and filling density, as per the design considerations that are treated later on (to be able to better grasp the performance details), note the possibilities of selecting different kinds (diameters and specific gravity) of various polyester filler materials. As said—it is important to notice that though outwards looks (appearance) of the Cough-EZ devices resembles some recognized and known face masks of prior art, the fact is that its construction, structure, properties, materials and contents, all intentionally designed, accounted for the beneficial results felt by Cough-EZ devices users as described and explained below), hence their function—namely their attributes and performance—achieve the sought for and promised result of soothing pain and irritation of all the entities involved in the unwanted coughing (throat, larynx, palate and other immediate adjacent organs of the body).

At the time the coughing bouts occur and result in pain and inconvenience, restoring to use the Cough-EZ devices results in shortening their duration, increasing the laps between following coughs while simultaneously reducing pain and inconvenience in real time as actually occurred—results that attests to the success of these innovative Cough-EZ devices.

To recapitulate—the Cough-EZ device was planned to introduce and provide three primarily functions.

They are—
1) Provide immediate, real-time relief while a person is actually coughing, resulting in an immediate lessening of pain and preventing the illness or irritation from worsening.
2) Coughing into the Cough-EZ would limit the spread of bacteria and microbes into the environment as well as kill some of the bacteria. It accomplishes this by having the Cough-EZ absorb most of the bacteria while antiseptic material in the device inner space will assist in annihilating (“killing”) attending pathogens.
3) Inhaling through the Cough-EZ limits (reduces) the amount of viruses and other air-borne pathogens (such as bacteria and microbes) the user would intake otherwise. It accomplishes this by having the Cough-EZ act as a fitter and its antiseptic material would assist in killing some of the bacteria. In this respect the Cough-EZ devices act similarly to the face masks (prior art) and might even be strapped by two cords temporarily over the mouth/nose area—but in contra-distinction to cited face masks serving only as filters, the Cough-EZ devices also continue to function in their primarily assigned task in accordance with the invention—devices acting to sooth and abate the pain and discomfort caused by the coughing bouts.
   It is important to realize that the immediate relief is obtained (to a certain degree) even without restoring to adding various substances (cited in Table No. 1)—but certain circumstances, for example allergy to the added lotions or even syrups, might necessitate the user to avoid using them. However, even in this case the user would benefit from the advantages provided by the Cough-EZ devices.

Let's conclude this summary chapter with the following important remarks and statements—emphasizing the following—

There are additional beneficial results to the three (3) enumerated above, providing added properties to the attributes of the Cough-EZ devices. They are:
a) The Cough-EZ is no less effective for sneezing at its different irritation and noise levels.
b) The Cough-EZ will also partly absorb, hence significantly reduce, the disturbing noise (harsh sound) associated with a cough (as well as a sneeze) so well known and sorrowfully familiar to concerts and theatre goers (especially, but not necessarily only in the winter time).
   The existence and level of the noise reduction features were actually tested and measured (see Table No. 3 and notes following it) and the results were found to strongly substantiate the above cited statement.
   Hence it is suggested and hoped, that Cough-EZ devices would be available for sale in Concert Halls and Theater foyers (lobbies), just as feature programs and hearing aids devices are nowadays available in these institutions.
c) The product is simple and requires no complex pharmaceutical or further technological development.
d) The Cough-EZ is relatively easy to manufacture, requiring simple materials and tools. Thus, it will be an inexpensive product to produce and purchase. It might well become an almost automatic addition to the standard medication basket for flues or colds in the average household.

To conclude this “Summary of the Invention” chapter, an alternative explanation that sheds light on the process steps that are the subject matter of the present invention, defined as “the essential trio behavior” effect—ARR—Absorbing, Rebounding and Relief, follows.

For ascertaining the successful fulfillment of the sought for real time Cough Soothing and Suppression immediate relief in accordance with the present invention, a sufficient and necessary condition is that the following Essential Trio steps: “Absorption—Rebound—Relief” would all be operating while a person is coughing—as explained and elucidated later. This means: (a) Absorption: absorbing and exploiting the energy of the cough, subject to using a calculated quantity of the selected absorbent polyester material in accordance with the invention design. See e.g. Wikipedia regarding the “cough force and speed”, and this concurrently with (b) below.

(b) Rebound: part of it is repelled and rebounds backwards, followed by (c)—
(c) Relief—relief is achieved in accordance with the present invention, as claimed thus providing “the Essential Trio”—as defined earlier when explaining the performance principle of the Cough-EZ soothing and suppressing devices (the cylindrical one and the pouch type one) and, as any professional would also understand, other configurations with similar structure and polyester fibers filling are within the scope of this application (note that “polyester fibers” is given solely as an example, and other fillings are also within the scope of this application).

We did not have tools to quantitatively measure the values of success, but qualitatively results in the field sustained and supported our claims.

Turning to a summary of the statements made above, let's elaborate on our definition of said essential trio. It hinges, as said, on exploiting the selection of the density and thickness of the polyester fibers as required for said “essential trio” combined with the related values of the components as given in tables No. 1 to No, 3.

For example, relating to the cylindrical device, we exploit the latent energy of the cough expelled (i.e., released) when coughing, as it was bound (closed within) the built in space of the devices.

As for the added substances—such as given in table No. 1, it is easy to understand that they contribute to enhancing the effectiveness of the performance of the structure (shape) of the “envelopes”—the elongated cylinder and the essentially semi-eliptic pouch devices, as stated above in this context.

### BRIEF DESCRIPTION OF THE ACCOMPANYING FIGURES

The present invention will be described hereinafter in conjunction with the accompanying figures. Identical components, wherein some of them are presented in the same figure—or in case that a same component appears in several figures, will carry an identical number.

FIG. 1. FIGS. 1a and 1b constitute illustrations presenting a Cough Soothing and Suppression device formed preferably as an elongated semi-cylindrical object (herein after—“a cylindrical Cough-EZ device”), also dubbed “the cylindrical device”.

FIG. 2 shows the preferred manner of holding the Cylindrical Cough-EZ device (shown in FIG. 1) opposite and near to the mouth, adjacent to the front (outwards) surface of the face.

FIG. 3 presents an illustration of an alternative Cough-EZ structure. A small semi-eliptic (pillow like) pouch constitutes a cough soothing device in accordance with the present invention.

FIG. 4 constitutes an illustration of the preferred manner of holding the pouch Cough-EZ device illustrated in FIG. 3, in accordance with the present invention.

FIG. 5 constitutes an illustration of a pillow like pouch Cough-EZ_{B} device, similar to the one illustrated in FIGS. 3 and 4, wherein its dimensions are extended to cover both the mouth and the nose areas (as shown), while there is a built in narrow tube extension formed in cited devices, so that part of said air burst from a cough is directed towards the nose nostrils section (not shown).

FIG. 6. FIGS. 6(a) to 6(c) depicts variations of face masks from the realm of “Prior Art”. They do not constitute a part of this patent application, but are presented herein for the completeness of our presentation.

FIG. 7. FIGS. 7(a) and 7(b), respectively, depict a-a cross section views of the cylindrical Cough-EZ device (illustrated in FIGS. 1 and 2). They present cutting patterns and arrangements of the polyester fibers in accordance with the invention and the design of the present patent application.

Any professional would understand that similar patterns of the polyester fibers are used also in the Cough-EZ pouch device (FIGS. 3 to 5). In order not to cloud the presentation with excessive figures, we refrain from presenting them here.

FIG. 8. FIGS. 8a and 8b, respectively, constitute cross section views of the cylindrical device, showing the inner back (rear) part of the device wherein it is strengthened in order to better prevent pathogens from being dispersed in the immediate vicinity of coughing persons. Its task doubles as a filter for the inhaling person's breathing. FIG. 8a shows this property being accomplished by an extra layer of cloth at the cylinder's back. FIG. 8b shows an alternate solution—achieving the same result by inserting extra and denser fibers of polyester near the rear inner surface. Similar layers are installed annexed to the back (rear surface) of the pouch device (not illustrated).

FIG. 9. FIGS. 9a and 9b, constitute cross section views of the Cylindrical and Pouch devices, respectively, illustrated as they are held flush near the front of the face mouth sector. They display the rushing outwards air-stream (cough) and the returned (echo like) cooler air flow, as marked by the arrows.

FIG. 10 shows a simple arrangement for preferably using a string or a cotton band to hang the device, so an (actually) coughing person can pick it up at ease and use it with no fuss when coughing is imminent (or occurring).

FIG. 11 shows an alternative and convenient mode for a person to carry a pouch on his wrist so it will be handy when coughing.

FIG. 12. FIG. 12a shows a cylindrical device wherein the space for the polyester fibers has a separable top cap attachable with a zipper or sew-on snaps. FIG. 12b shows a pouch device wherein the space for the polyester fibers has a separable (semi-) elliptical separate front surface, attachable similarly with a zipper or sew-on snaps.

FIG. 13. FIGS. 13a to 13f, are given as a summary, to recapitulate the various views, explanations and entities annexed to the cylindrical Cough-EZ device, in the former exposition.

FIG. 14 shows an additional version of said cylindrical Cough-EZ device, wherein the user can control the intensity of the returned “echo” (and also level of energy absorption) by contracting (thus shortening) the length of the lengthwise dimension of the device—and letting go for returning to its original length (“height”).

### DETAILED DESCRIPTION OF PREFERRED EMBODIMENTS

In the following description, various aspects of the invention will be described. For the purpose of explanation, specific configurations and details are set forth in order to provide a thorough understanding of the techniques. However, it will also be apparent to one skilled in the art that the techniques may be practiced without specific details being presented herein. Furthermore, well-known features may be omitted or simplified in order not to obscure the description of the techniques.

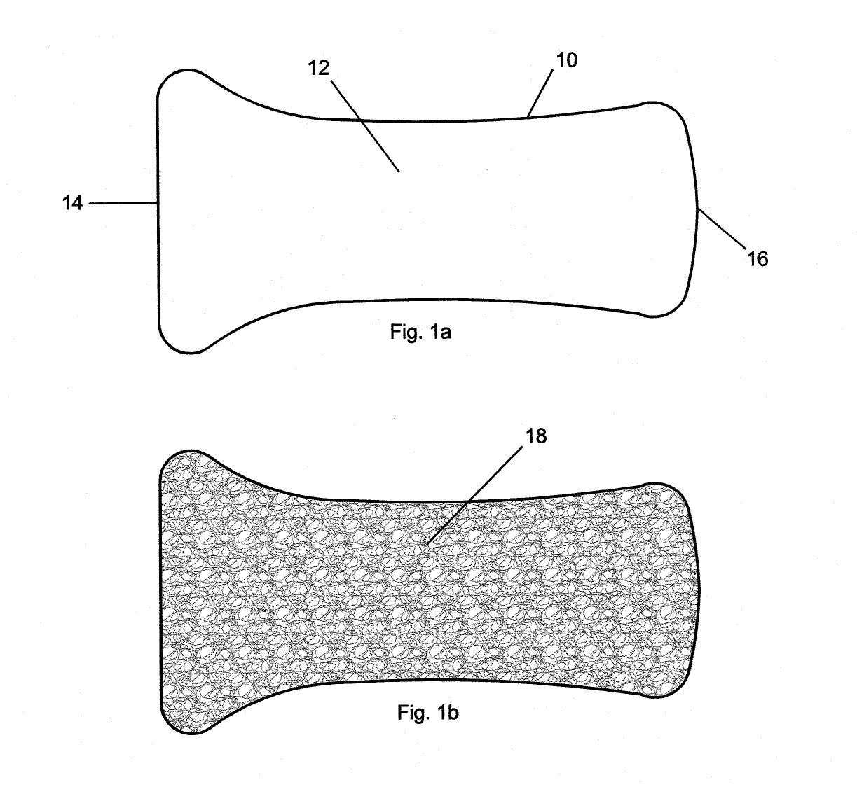

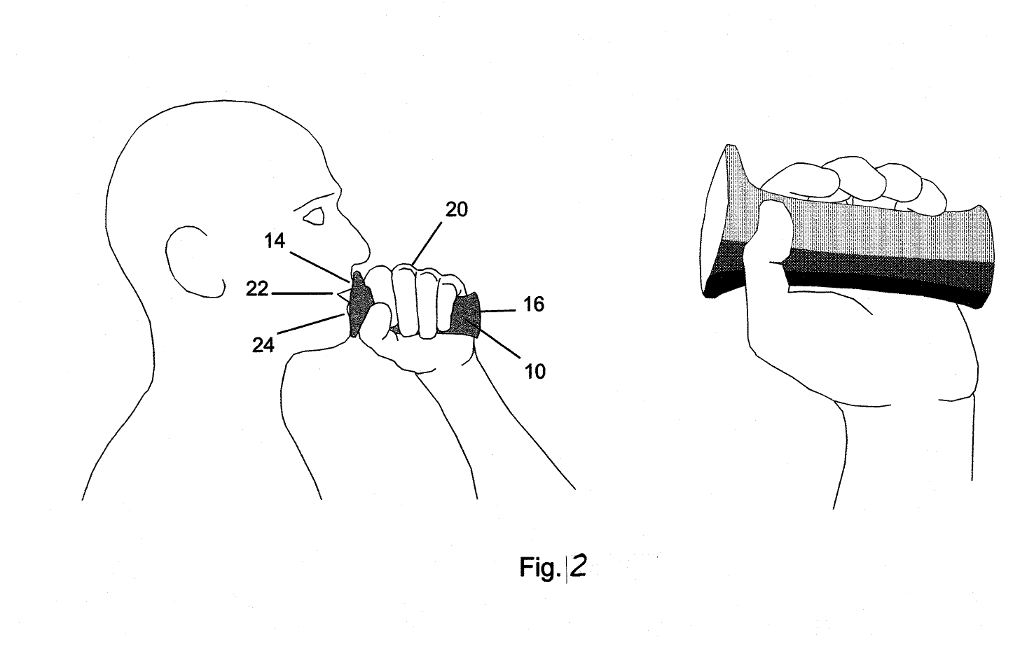

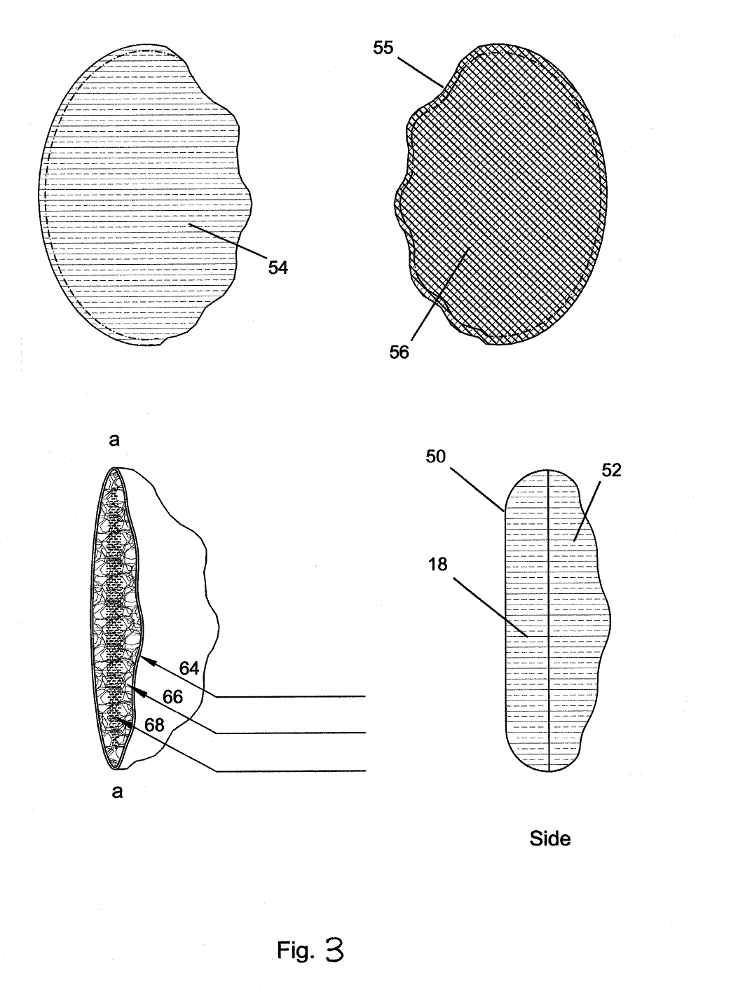

Reference is being made to FIGS. 1 to 4. FIGS. 1 and 3 constitute illustrations of two various embodiments of Cough-EZ devices, that constitute the subject matter of the present patent application.

FIGS. 1a and 1b constitute an illustration presenting a Cough Soothing and Suppressing device 10 that is formed preferably as an elongated cylindrical object (hereinafter—a “cylindrical Cough-EZ device”) also dubbed “the cylindrical device”. It is preferably made of cloth, or Lyocell synthetic fiber stitched to form a space 12 that is preferably filled up with polyester fibers 18. It is to be remembered that polyester fibers are cited as appropriate filling material—but only as an example, and other materials are used too, provided they have more or less the same properties that are suitable to provide the requirements (availability, strength, absorption etc.), as cited wherein it is recounted in the text.

FIG. 1a presents the outline of the device; FIG. 1b—shows the cross-section of the device 10 as it is filled with polyester fibers 18.

FIG. 2 shows the preferred manner 20 of holding the cylindrical Cough-EZ device 10 opposite and near to—, flush touching—the mouth 22, at the front (outwards) surface 24 of the face.

The shape of the cylindrical Cough-EZ device 10 was designed to make it hand-friendly, namely convenient and easy to handle—to be brought correctly flush-next to the mouth/face outwards surface (see figure) with intervals whenever beneficial, held so that its upper circular (round) top 14 is held next to and facing the mouth 22 (see FIG. 2). namely—the circular top 14 is brought to cover the mouth and chin face sector 24, as shown. Structure, materials and properties of the rear surface (disk 16) would be presented herein below.

Let's refer to FIG. 3. FIG. 3 presents an illustration of an alternative Cough-EZ structure. It is a small semi-eliptic item that constitutes the pouch Cough-EZ device 50. Its dimensions and form (including axes and directions) differ from the cylindrical device 10 shown in FIG. 1, but in principle this Pouch 50, is likewise preferably made of cloth or Lyocell synthetic fiber stitched to form a space 52 to be preferably filled up with polyester fibers 18, wherein the polyester is preferably “soaked” in or sprayed upon with specific liquids as said (see also Table No. 1). Precisely, by the term “soaked” it is meant that the polyester, on producing the devices, is soaked in (or sprayed upon) with the selected fluid is soaked as per Table No. 1 preferably with a dose of antiseptic mixed in the solution, then pressed as said so that the polyester fibers adsorb the selected preparation until they are actually dry on the outside, while the beneficial desired aroma/fumes is felt and continues to be effective, in addition to the contribution of the effect of the essential trio process (ARR—Adsorption, Rebound, Relief) as explained elsewhere in detail. Note that instead of using various fluids, it is feasible to insert rigid granules such as menthol or eucalyptus, leading to the same beneficial results as achieved by the above cited fluids (table No. 1).

Note that FIG. 3a shows an a-a section of the pouch, presenting the outer fabric layer 64, the absorbent material 66 and the antiseptic material 68.

Similarly, the back (rear) surface area 56 has similar properties and options of material as the rear disk 16 of the Cylindrical device 10. The two sides 55 complete the pouch (in production—they constitute an integral part f the envelope of the pouch).

TABLE NO. 1
Recommended Additives to Soak the Polyester Fibers

| | Ingredient | | | | |
|---|---|---|---|---|---|
| | | | | Granules | |
| Additives | Capsules | Syrups | Oils | Solids | Spray |
| Medications | Codeine | Codeine | ** ** | Sinusin- | ** ** |
| | Codivis | Codivis | | Heel | |
| Homeopathic/ | ** ** | Bronchomel | 1. Eucalyptus | 1. Eucalyptus | Spray |
| Etheric/ | | | 2. Menthol | 2. Menthol | propolis |
| aromatic | | | | | |
| Syrups and | ** ** | 1. Prothiazine | ** ** | ** ** | ** ** |
| medical | | expectorant | | | |
| syrups | | Syrup | | | |
| | | 2. Tussosedan | | | |
| | | 3. Toplexil Syrup | | | |

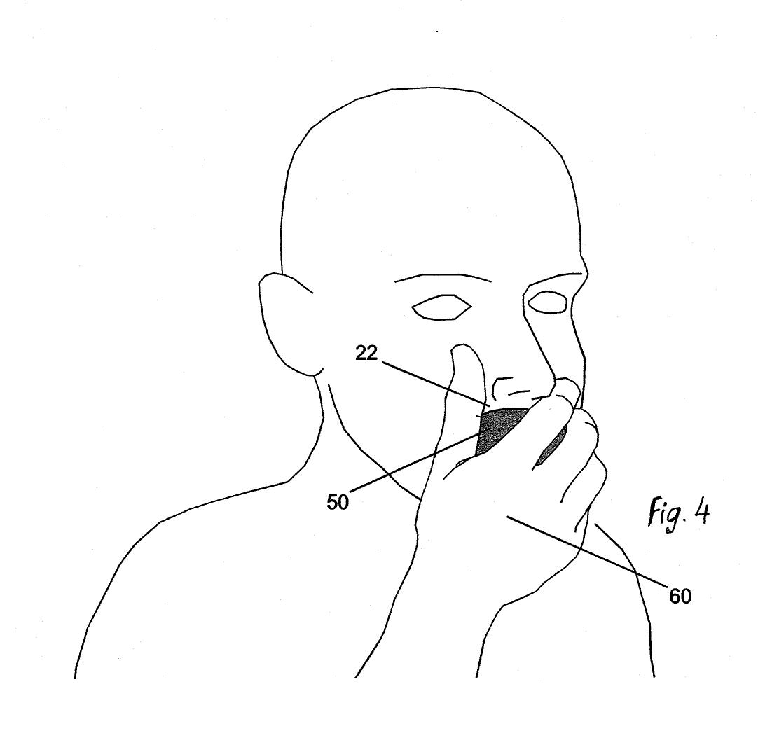

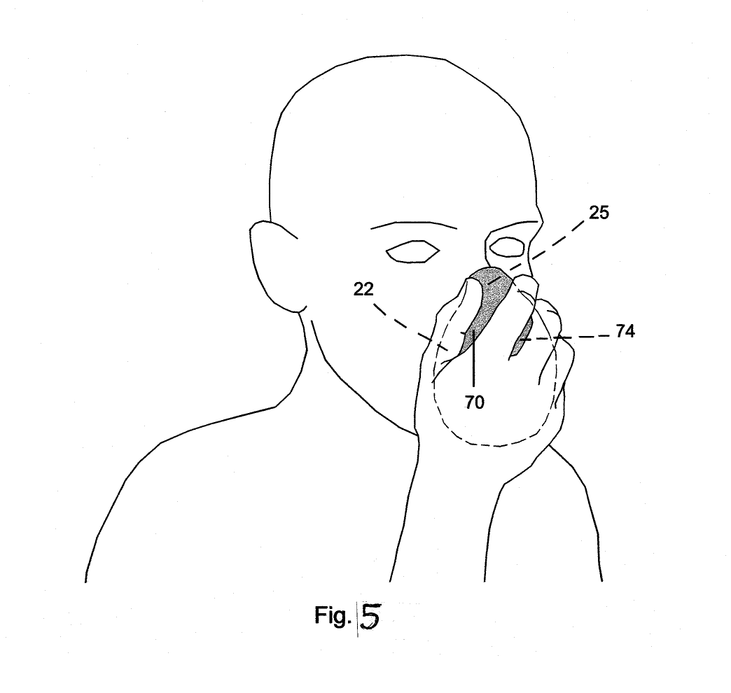

Let's revert to FIG. 4. FIG. 4 constitutes an illustration of the small pillow like pouch Cough-EZ device Cough Soothing and Suppressing device 50 (in short: “Pouch Device”) illustrated in FIG. 3, wherein it shows the manner 60 of holding flush said pouch Cough-EZ device 50 against the mouth's face sector 24.

The pouch is held so that its front area 54 faces (covers flush) the mouth, whereas the hand covers the rear surface 56 providing extra ability to prevent exit of spit, pathogens and other ingredients harmful to the neighborhood when coughing. Note the manner 60 of holding the pouch (it is similar to the manner 20 of holding the cylindrical Cough-EZ device 10—see FIG. 2) wherein the mouth 22 is facing front area 54 of the pouch. The properties of the rear surface 56 of the pouch Cough-EZ device are similar to those of the rear disk 16 of the cylindrical device, it helps the rebound property and the soothing effect, as well as being an added two way filter vs. the environment).

We emphasize that not withstanding the difference in structure, the cylindrical and the pouch devices have similar attributes and same operational modes (features). In addition to the basic features that were enumerated and discussed above and herein below (namely cough soothing, real time relief and filtering pathogens and other non welcome environmental negative constituents) they have additional beneficial attributes that are inherent in the invention.

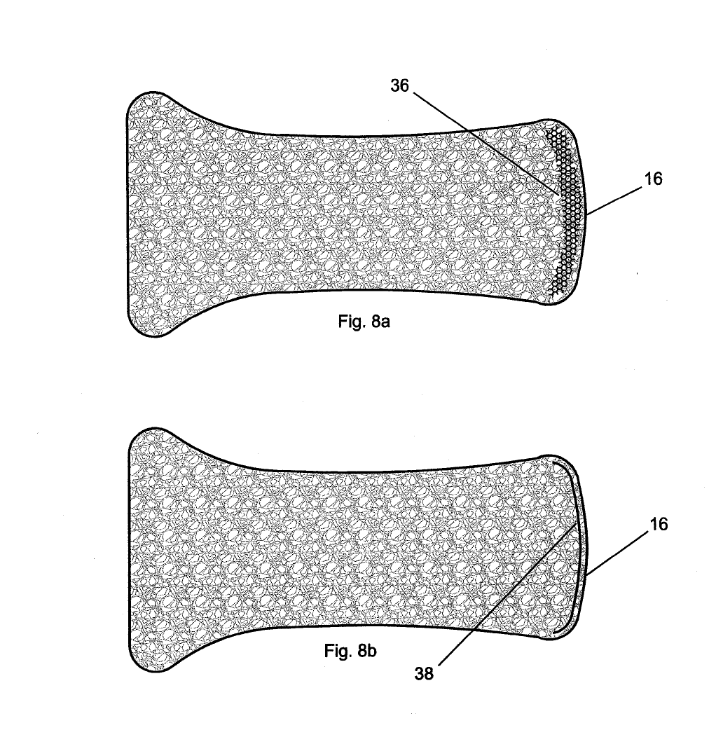

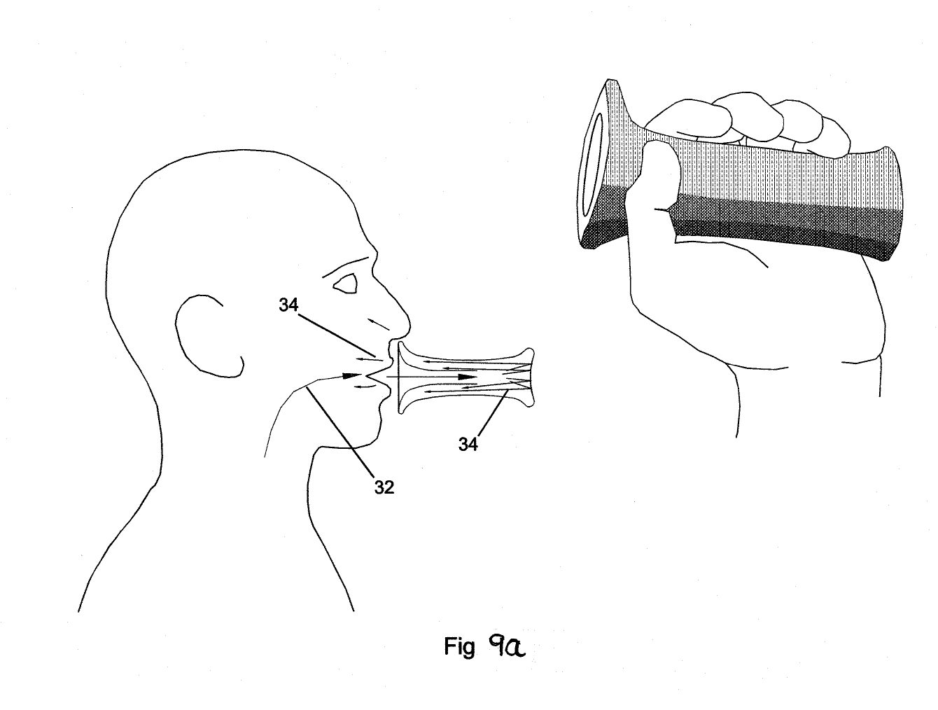

The first one is that the rear (back) surface 56 of the pouch is strengthened by an additional denser polyester layer inside—next to it, or by an extra non permeable cotton cloth or non-woven layer (similar to 36 and 38, respectively, in the cylindrical device—see FIGS. 8a and 8b). A variation of the cloth backing, which improves the performance of the devices, is gained by using for this added layer a “Lyocell” synthetic fiber which provides an increased capability to prevent pathogens (microbes, germs etc.) from passing through it into the environment (while simultaneously improving the devices' performance).

Moreover, there exists an additional reason (that contributes to upgrade the soothing performance) for strengthening the back surface 16 in the cylindrical device and 56 of the pouch, respectively. It is that in the scale of the rebound property of the essential trio, the extra polyester fibers strengthens the rebound capability (its contribution to the performance was explained above). As a synergism, it also enhances the filtering results (no pathogens exiting to the environment nor entered when inhaling).

The influence on the soothing and suppression performance is anticipated to improve due to the structure and the polyester (plus by soaking in selected solutions, as said).

The physiologic contribution (in accordance with the present invention) to the achieved real time soothing and suppressing of the cough bouts (the discomfort as well as the frequency) would be elucidated below—on presenting the justifying evidence to the statement above (i.e., “the essential trio” process).

Similarly to the cylindrical device 10 discussed above, also the smaller pouch cough-EZ device 50 was designed to make it hand-friendly, namely convenient and easy to handle—to be brought forth correctly, laid opposite and flush with the mouth (i.e., the face outwards surface 24—see FIG. 4)—with intervals between applications whenever beneficial, held so that its semi-eliptic front surface 54 will be held next to—and flush with—the mouth 22—namely, the semi-eliptic front area 54 is brought to face the chin (mouth's face sector 24), as shown. The smaller and less bulky pouch Cough-EZ device 50 (not as the cylindrical Cough-EZ device 10) can be conveniently carried in a gentleman's pocket or even a lady's small purse—nestled in their sealed (impermeable) plastic bag 110 (not shown).

Let's revert to FIG. 1. The dimensions of the various parameters (diameter, height etc.) are presented in table No. 2, together with other relevant data, such as the material of the envelope, the weight and density of the filling polyester fibers and so on—and together with the same “parallel” data applying to the pouch Cough-EZ device illustrated in FIGS. 3 and 4.

Any professional would appreciate the fact that selecting the size, density and weight of the polyester fibers, combined with the shape of the outlines (envelopes) of the cylindrical and pouch Cough-EZ devices, contribute to their properties and attributes—providing real time help and relief from the pain and irritation suffered when coughing—these flavors add significantly to the immediate effect of soothing and suppressing the cough, but also because of the antiseptic qualities of some of the added solutions that protect the user (as well as his vicinity) from all kinds of pathogens and malicious microbes and germs. People with allergies to the flavor materials (table No. 1) can resort to using solid granules (Eucalyptus, Menthol), getting the same cool, fresh intake and real time cough soothing results.

Any professional would also understand that the configuration of the Cough-EZ devices (cylindrical 10, pouch 50), the polyester fillings 18 (with their preferably selected weight, shape and density) that are illustrated in the figures and were described above, might also be in different and various shapes, for example a small circular cylinder (with its diameter just fitting to cover the mouth's face area as said and other different fitting shapes). Similarly, any professional would also understand that the added medical, homeopathic and other ingredients might be replaced by other substances—endowed with similar attributes and hence providing similar results (within the scope of the present patent application).

TABLE NO. 2
Dimensions and Other General and Relevant Technical Data*

| | | | Absorbing | Material's |
|---|---|---|---|---|
| | Dimensions | Envelope | Material | Amount (gram & |
| Shape | (cm) | Materials | (Density-Denier) | density) |
| Cylinder | Diameter: 5 | Cotton 100%\| | Polyester\|6.0 | 20-6.0 den |
| Covers Mouth | Height: 13 | Cloth: 127/79 | | |
| | | (Satin) | | |
| Larger Cylinder | Diameter: 7.5 | Cotton 100%\| | Polyester\|6.0 | 21-6.0 den |
| Covers Mouth/nose | Height: 13 | Cloth: 127/79 | | |
| | | (Satin) | | |
| Pouch, flat, semi- | 9.5 × 11 × 5 | Cotton 100%\| | Polyester\|0.8 | 10-0.8 den |
| elliptic, Covers | | 76/68 | | |
| Mouth | | | | |
| Pouch, flat, | 9.5 × 14 × 5 | Cotton 100%\| | Polyester\|0.8 | 12-0.8 den |
| semi-elliptic, | | 76/68 | | |
| larger- | | | | |
| Covers Mouth/nose | | | | |

*This table represents sets of values used for one specific exemplary evaluation, other materials and dimensions were also checked.

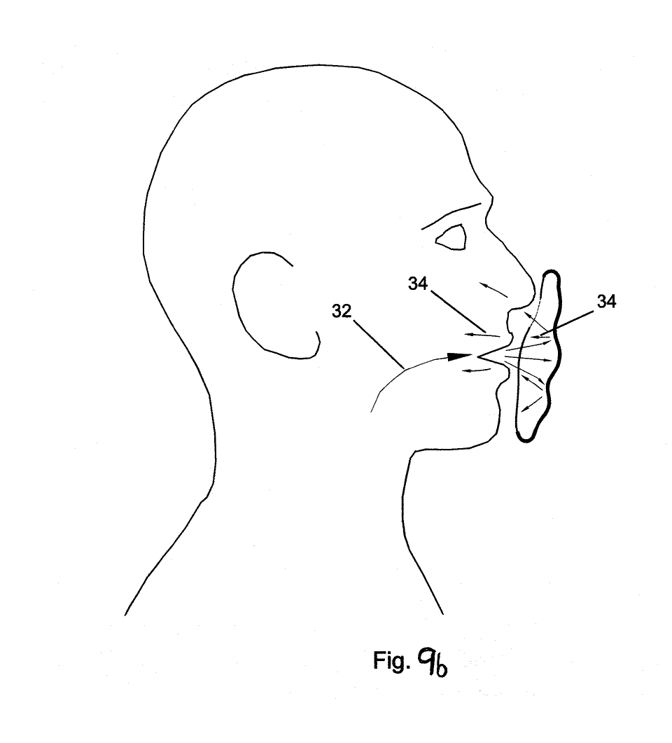

Any professional would appreciate the fact that selecting the size, density and weight of the polyester fibers, combined with the shape of their outlines (envelopes) of the cylindrical and pouch Cough-EZ devices, contribute to their property (attribute) of providing real time help and relief from the pain and irritation suffered when coughing. This is explained below, when referring to the rushing air flow when coughing and its effects—see the text associated with FIG. 9.

The reason (justifications) for selecting the various materials that are used, their densities—and as for the polyester—the way of inserting them, the selection of different weights and “crowding” (thus selected density)—all of which contribute to achieving the designed performance of the Cough-EZ devices. The considerations include the distance that the cough passes within the device before exiting to the environment, in order to achieve the “essential trio behavior” effect of absorbing, redounding and relief as explained elsewhere in detail.

On the one hand—in the cylindrical device, whose height is 13 cm, the polyester fibers for fulfilling the relief goal is 6.0 denier.

On the other hand, as for the pouch device, said “distance to be passed” for the air flow being absorbed is shorter (6 cm)—hence a larger crowding of the fibers is used for obtaining the same result with the “essential trio behavior” (effect of absorbing, redounding and relief). In both cases—the results were tested and verified in clinical surroundings.

When coughing, the upper surface—disk 14, of cylindrical device 10; and similarly the front semi-eliptic area 54 of pouch device 50 (namely the area that would be opposite mouth's front area sector 24) respectively (see FIGS. 2 and 4) serve as said to accomplish three major functions that constitute the subject matter of the invention and justification of the present patent application. Namely—
(a) reduces (diminishes) in real time—the pain, itch and discomfort in the throat and larynx t (also treats itching in the palate) associated with involuntary coughing bouts (involuntary “efforts”) caused by coughing, in accordance with the present invention, and—
(b) by abating the coughing itch and attendant pain in the throat and palate, influences the involuntary coughing bouts so that in real time—the time between successive coughing bouts and bursts increases, just adding to the feeling of relief and reduced pain caused by the involuntary successive coughing bouts, and—
(c) traps, within said polyester filling and abolishes (“kills”) some of the pathogens (microbes, germs, etc.) discharged (or even spitted) from one's mouth, thus preventing them from being dispersed in its surrounding environment or even landing in the adjacent environment or on a nearby persons' face/body; and—concurrently it acts as a face mask—but these Cough-EZ devices provide more benefits (soothing action) then just “filtering the air”, by achieving real time soothing and suppression of the pain and irritation that comes with the coughing—it achieves relief in accordance with the present invention, as explained here to fore and later on (below).
(d) from a study of the results (in real time) presented above, we found—as said, a synergism that stems from the structure and materials (polyester fibers) making up the Cough-EZ devices—namely: the sounds (noise) of the coughs (and sneezes, where applicable) are essentially “blocked”, for instance—the difference between 60 db to 63 db is twice as much and vice versa, thus contributing to the well being of people around the coughing person (imagine the “winter concerts” of coughs in concert halls and theatres). We also mention (prior art discussion) the McBrearty's U.S. Pat. No. 5,413,094—granted for a noise reducing mask (“A No Noise Apparatus face mask”) implemented by a face mask containing layers of noise absorbing acoustical substances. In this case, a person has to be a very considerate person (or one that does not wish to be a coughing noise source attracting attention and scolding looks in public places—see other examples presented in McBrearty's patent). In our case, the noise abating property is an inherent attribute of the Cough-EZ cylindrical and pouch devices, in accordance with the present invention, as said, due to the polyester fibers and envelope, and the act of covering the mouth when coughing.
   In table No. 3, we present quantitative values of the noise reduction levels achieved by the line of Cough-EZ devices (data not given in the cited patent). We also measured for comparison—how much do regular paper, cardboard or fabric face masks (prior art) reduce the noise—and it is much below par as compared to the cough easy devices.

In summary, the orderly, quantitative experiments and tests—that were carried out by us, using standard processes of the acoustics-physics discipline, in an echo free room, subject to conducting the measurements under the accepted standard value of reverberation time=RT₆₀ (for a definition—consult Wikipedia). The reverberation time can be modeled to permit an approximate calculation.

The “Range” decay was=70 dB

TABLE NO. 3
Results of Measured Noise Reduction Values (Tested)

| | | Measurement | Measurement | |
|---|---|---|---|---|
| Shape of the | Background | without the Cough- | with the Cough-EZ | |
| Cough-EZ device | Data | EZ device″ | device | Remarks |
| Cylinder | See background | 85 dB | 68 dB | — |
| | data below | | | |
| Pouch (flat) | See background | 85 dB | 68 dB | Measurement |
| | data below | | | without the HAND |
| | | | | covering/holding |
| | | | | outside. |
| Pouch (flat) | See background | 85 dB | 66.9 dB | Wherein the HAND |
| | data below | | | covers the Cough- |
| | | | | EZ device at the |
| | | | | back |
| Small Flat | See background | 85 dB | 67 dB | Covers Mouth only |
| Pouch | data below | | | |
| Face Masks - | See background | 85 dB | 84 db | — |
| paper, cardboard | data below | | | |
| or fabrics | | | | |

Test background data:
Range: 70-130 SPL;
Weight: A;
Value: RT₆₀
Room RT₆₀: 0.7 sec;
Decay range: 70 db

Reference is made to FIG. 5. FIG. 5 constitutes an illustration of another semi-elliptical pouch Cough-EZ_{B} device 70, larger than the one depicted in FIGS. 3 and 4. Its dimensions are larger (extended) as compared to the previous values, so that it is, as said, large enough to cover the mouth 22 and the nose 25 areas jointly, thus in this case the nose would also be completely facing (and flush touching) the front surface area 74 of the pouch Cough-EZ_{B} device.

Any professional expert in the field, would appreciate the fact that this Cough-EZ_{B} device might improve the Cough Soothing and Suppressing performance (due to the larger volume of the present polyester fibers), and moreover, the emitting of malignant entities (microbes, germs and mucus etc. when sneezing would be eliminated. Simultaneously, the noise (sound) of the sneezing act would be reduced—a blessing to the environment, especially in closed quarters.

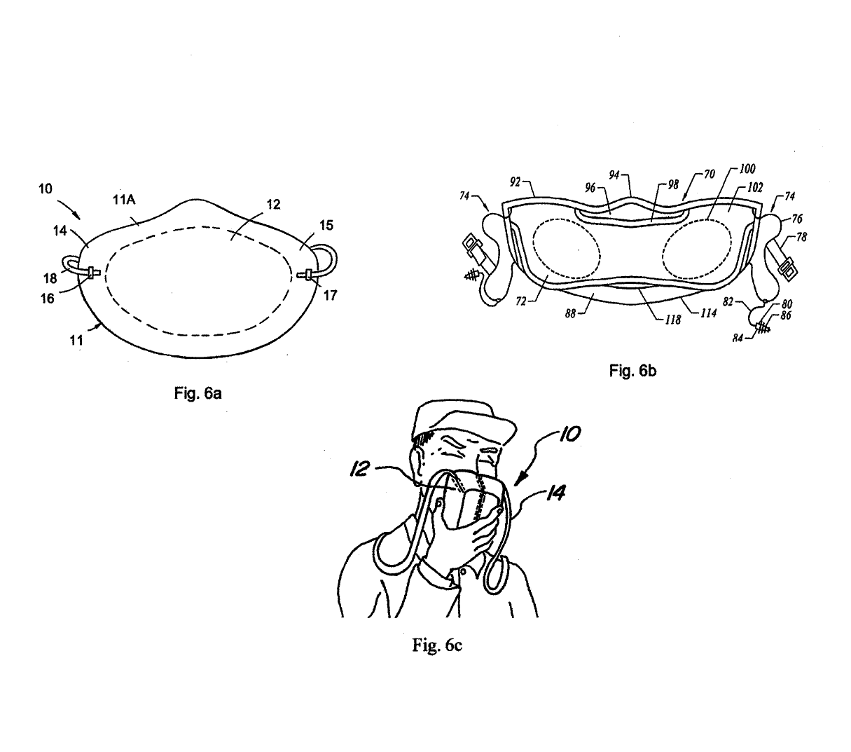

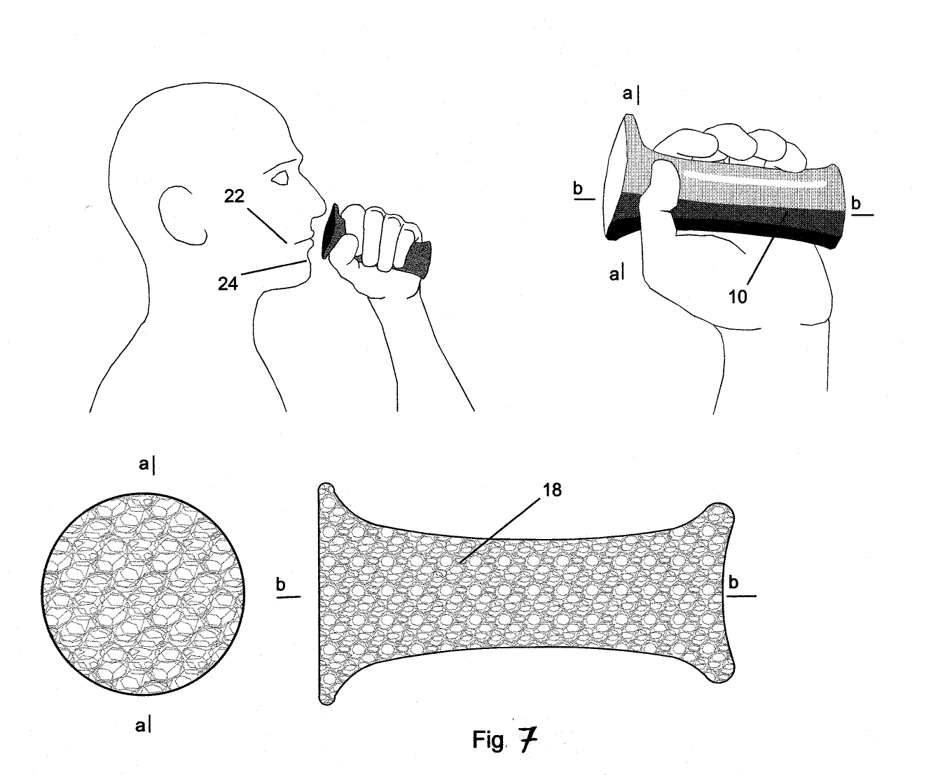

Let's refer to FIG. 6. FIGS. 6 (a to c) are from the realm of “Prior Art”. They are included in the presentation of the figures as said, but do not constitute a part of this patent application. They are solely presented for the completeness of this application—included in order to substantiate the statements made above relating to the benefits of the Cough-EZ (& EZ_{B}) Soothing and Suppressing devices vis a vis the multitude of face mask patents which differ one from the other in small details, but serve only as filters (in both directions) against spreading pathogens by coughing on the one hand and simultaneously protects from inhaling them and other malignant ingredients from the surrounding environment on the other end. Adding antiseptic material, as said, to be adsorbed in the polyester fibers—upgrades the protection against pathogens provided by the Cough-EZ devices. These figures illustrate, as in FIG. 6a, a “Face Mask and method”, U.S. Pat. No. 4,856,509 (Jerome H. Lemelson), in FIG. 6b a “Travel Mask”, U.S. Pat. No. 6,823,868 (Paul G. Begum) and in FIG. 6c with a different approach—McBrearty's U.S. Pat. No. 5,413,094 granted for a noise reducing mask (“A No Noise Apparatus face mask”) implemented by a face mask containing noise absorbing acoustical substances.

In the text of McBrearty patent we did not find numerical data (values) quoting the quantitative reduction of the coughing/sneezes noises by that apparatus, information that is given in our Table No. 3, attesting to the effectiveness of the Cough-EZ devices also in this aspect.

Let's refer to FIG. 7. FIGS. 7(a) and 7(b), respectively, depict a-a cross section views of the cylindrical Cough-EZ device (illustrated in FIGS. 1 and 2). All the data, information and descriptions relating to the cylindrical device, apply and hold also, mutatis mutandis, to the pouch Cough-EZ devices (regular and extended, FIGS. 3 to 5), wherein cutting patterns of the polyester fibers are shown, as said, with their respective polyester fillings, that intentionally vary in their specific weight, density and size values—in accordance with the design requirements of this patent.

Note the data about the preferably polyester filling parameters presented in table No. 2 (and the comments following it). We are using various medical, homeopathic and also antiseptic solutions (see table No. 1) for soaking the polyester fibers (or by spraying on them these solutions from the outside, using a dedicated apparatus). In other words: the polyester filling would be preferably soaked in—or wetted upon by—various solutions, but then pressed as said so that the liquids are absorbed by the polyester fibers. To enhance the merits of the polyester fibers that absorbed the various lotions (and pressed so that they are essentially dry on the outside). We refer to the data and information given above in respect to the effectiveness of soothing and abating the pain as stated—as well as the data relating to the use of added analgesic substances and related options in accordance with the invention as they were explained above and repeated herein below.

Let's refer to FIG. 8. FIGS. 8a and 8b, respectively, constitute cross section views of the cylindrical Cough-EZ device, showing the inner back (rear) part of the device wherein it is strengthened (in order to better prevent pathogens from being dispersed in the immediate vicinity of coughing persons environment) by an added fabric layer 38. In other words: these caps are introduced to improve (upgrade) the prevention of spreading pathogens and other non-desired substances to the environment either by one or more layer/s forming a “cap” 36—made of semi-impenetrable cloth (FIG. 8a) or by inserting a more dense layer of polyester fibers 38 flush with the inner rear surface (FIG. 8b).

Their action fulfills two fold goals, namely improve insulation (in and out) of pathogens, and also enhance the rebound property (hence—performance) of the essential trio.

Any professional would understand that similar strengthening of the rear inner surfaces, is also done and effective in the case of the semi-elliptic pouch—with either semi-impenetrable cloth or denser polyester layer (not shown).

Let's refer to FIG. 9. FIGS. 9a and 9b, respectively, constitute cross section views of the Cylindrical and Pouch devices as they are held near the front of the face, showing the “flowing” streams of air (outwards due to the coughing, and “rebound”, i.e. returned inwards like an echo—in accordance with the properties of the devices. They are shown by arrows 32 and 34, respectively. The flow (stream) of air 32 from the mouth and larynx rushing outwards, being expelled by the cough, is approximately at the temperature of the closed space it is in—namely the body temperature. As it rushes outwards, it expands and hence its temperature drops, thus delivering a cooling, pleasant caressing gust of air 34, even more so due to the fumes of the ingredients we introduced into the polyester fibers. The temperature decreases even more—because the atmosphere around is normally several degrees lower (more so in the winter). This cool air is being returned to the mouth that is still open, now bringing a cooler air flow which is manna to the sore organs subjected to the cough irritation. Furthermore, the air adsorbs some of the components of the aromatics, medical or homeopathic ingredients and other substances in the soaked polyester fibers, thus providing a pleasant and soothing air stream, a blessing to the coughing person, that influences even toddlers and kids.

Similar pleasant relief is also felt if the liquids are replaced by solid granules providing the same cool feeling, such as eucalyptus or menthol pellets.

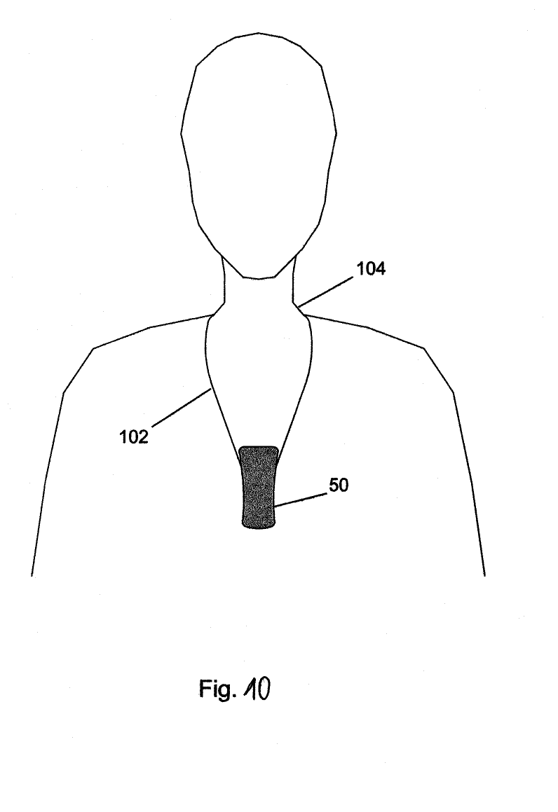

Let's refer to FIG. 10. FIG. 10 shows a simple arrangement for preferably using a string, a cotton band or a rubber band 102 over the head and on the nape 104 to hang the device 50, so an actually coughing person can pick it up at ease and use it with no fuss when coughing is imminent (or occurring).

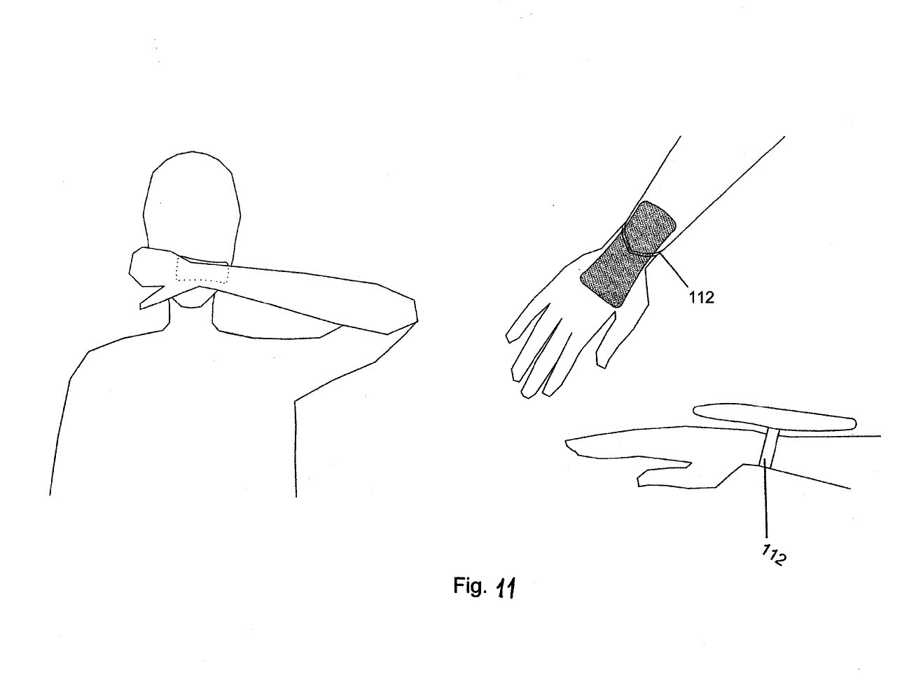

FIG. 11 shows an alternative and convenient mode for a person to carry a pouch 10 so it will be handy to be used in case of coughing. It is embodied by a wide rubber band 112 or a nice fancy strap 114 (that is not illustrated).

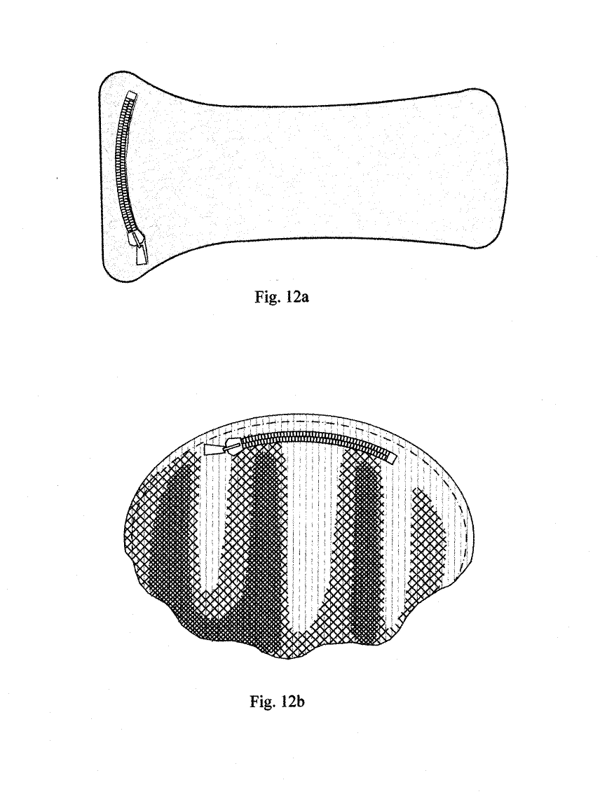

FIG. 12. FIG. 12a shows a cylindrical device 10 wherein the space 12 for the fibers 18 has a separate top cap 15 attachable with a zipper or sew-on snaps so that the user can open and refill she space 12 at will (insert granules or fluids over the polyester fibers).

FIG. 12b shows a pouch device 50 wherein the space 52 for the fibers 18 has a separate front surface semi-elliptical separate surface 55, attachable with a zipper (or sew-on snaps), so that the user can open and refill the space 52 at will (granules or fluids over the polyester fibers).

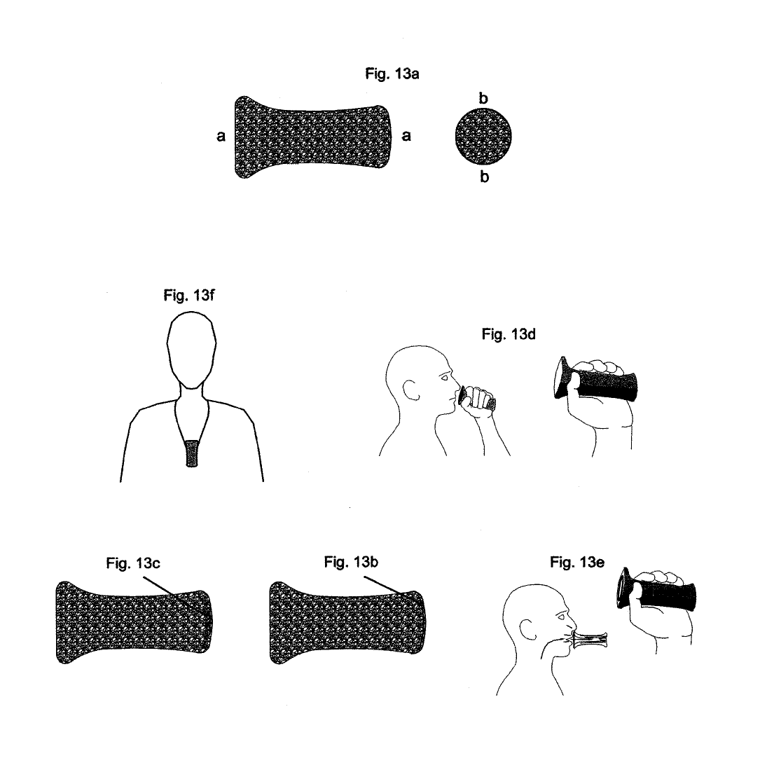

FIG. 13. FIGS. 13a to 13f, are given as a summary, to recapitulate the various views, explanations and entities annexed to the cylindrical Cough-EZ device, in the former exposition.

Thus—

FIG. 13a shows the cylindrical device (a-a length-wise and b-b “disc” cross sections) showing the polyester distribution.

FIG. 13b shows the Cylindrical device wherein the rear (back) wall is reinforced by a cloth disc.

FIG. 13c shows the Cylindrical device wherein the density of the polyester fibers at the back volume is increased.

FIG. 13d shows the device as it is held facing the chin while holding it flush over the mouth.

FIG. 13e shows the device as it is held facing the mouth and the flow of the air streams—outwards and returned (rebound, as said), that is essential for understanding the soothing effect inherent in the present invention.

FIG. 13f shows an arrangement for hanging the device with a band over the head and nape—(see FIG. 10) so it will be handy to use when coughing.

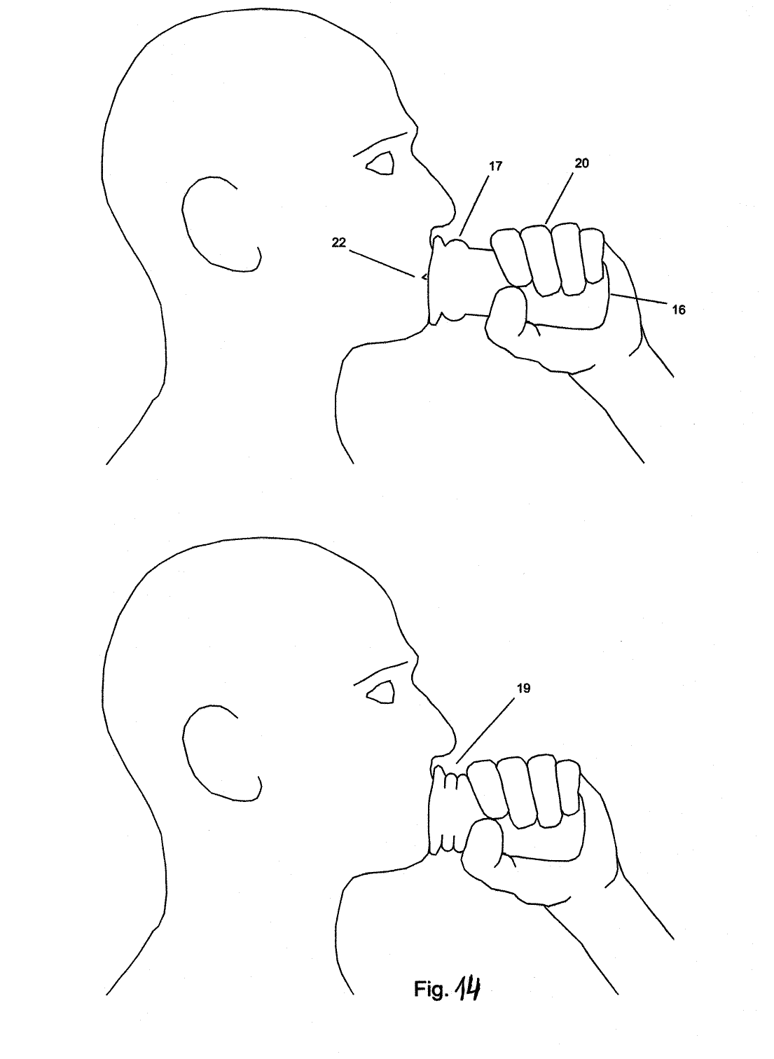

FIG. 14 shows an additional version of said cylindrical Cough-EZ device, as defined in FIG. 2, wherein the user can control the intensity of the returned “echo” (and also level of energy absorption) by contracting (thus shortening) the length of the lengthwise dimension of the device 17 (or 19 for further contraction)—and letting go for returning to its original length (“height”).

Any professional would understand that the present invention was described above only in a way of presenting examples, serving our descriptive needs. Thus, any changes or variants in the structure of the devices aimed at reducing pain and discomfort linked to or associated with coughing spells—the subject matter of the present invention, would not exclude them from the framework of this invention.

In other words, it is feasible to implement the invention as it was described above while referring to the accompanying figures, also with introducing changes and additions that would not depart from the constructional characteristics of the invention, characteristics that are claimed herein under.

1-27. (canceled)

28. A device designed to reduce the noise, pain, itching, and discomfort associated with involuntary coughing and sneezing attacks and to bring these attacks to an end, said device comprising an envelope, said envelope comprising:
a. a front surface configured to be held flush with a portion of a face;
b. a back surface; and
c. a hollow interior filled with material adapted to reduce the speed of the stream of
air expelled from the mouth or nose and the noise associated with a cough or sneeze; characterized in that the back surface is strengthened by adding, flush with the interior of the back surface, at least one of: (a) a fabric layer of semi-impenetrable cloth and (b) a dense layer of polyester fibers; thereby creating a surface from which a portion of the stream of air expelled from the mouth or nose during a cough or sneeze rebounds and is returned towards the mouth or nose and through which a portion of the stream of air exits the device allowing normal breathing when the device is held flush with the portion of the face.

29. The device of claim 28, wherein the portion of the face is one of: the area surrounding a mouth, the area surrounding a nose, and the area surrounding both a mouth and a nose.

30. The device of claim 28, wherein the shape of the envelope is selected from: a cylindrical shape and a pouch shape.

31. The device of claim 28, wherein the envelope is made from cloth or Lyocell synthetic fiber.

32. The device of claim 28, wherein the envelope is filled with polyester fibers.

33. The device of claim 30, wherein the cylindrically shaped envelope is filled with 6.0 denier polyester fibers.

34. The device of claim 30, wherein the pouch shaped envelope is filled with 8.0 denier polyester fibers.

35. The device of claim 28, comprising a spray or a solution comprised of at least one medication, homeopathic substance, and syrup absorbed in the material adapted to reduce the speed of the stream of air expelled from the mouth or nose and the noise associated with a cough or sneeze, wherein the aroma of the spray or solution mixes with the portion of the stream of air expelled from the mouth or nose that rebounds from the back surface and returns towards the mouth or nose; thereby soothing and abating the pain of the cough or sneeze.

36. The device of claim 28, comprising solid granules of aromatic material in the hollow interior of the device, wherein the aroma of the spray or solution mixes with the portion of the stream of air expelled from the mouth or nose that rebounds from the back surface and returns towards the mouth or nose; thereby soothing and abating the pain of the cough or sneeze.

37. The device of claim 28, comprising a zipper or sew-on snaps allowing the hollow interior of the device to be opened and refilled.

38. The device of claim 28 configured such that the user can change the length of the device to control the intensity of the portion of the stream of air that rebounds from the back surface and returned towards the mouth or nose.
